(19) United States
(12) Patent Application Publication
(10) Pub. No.: US 2013/0164265 A1
(43) Pub. Date: Jun. 27, 2013

(54) SKIN CARE COMPOSITIONS

(71) Applicants: Dana FLAVIN, Starnberg (DE); Sigrid OBENLAND, Munich (DE)

(72) Inventors: Dana FLAVIN, Starnberg (DE); Sigrid OBENLAND, Munich (DE)

(21) Appl. No.: 13/333,303

(22) Filed: Dec. 21, 2011

(51) Int. Cl. A61K 8/97; A61K 36/71; A61K 36/481; A61K 8/63; A61K 36/48; A61Q 19/00; A61P 17/00; A61K 31/704; A61K 36/00

(52) U.S. Cl. 424/93.7; 514/26; 424/725; 424/773; 424/757

(57) ABSTRACT

This invention provides compositions and methods of manufacturing such compositions that employ GRAS compounds which can act to promote the generation of stem, epidermal or other skin cells in the epidermis, activation of collagen synthesis, activation of hyaluronic acid synthesis, enhanced skin hydration, and dermal healing by stimulating stem cell and fibroblast migration to sites of needed repair. These compositions and methods are useful for rejuvenating the skin and for treating some skin-related aging or other dermal damaging conditions, including wrinkle reduction and treatment of minor dermal wounds.

### BACKGROUND OF THE INVENTION

1. Field of the Invention

The invention disclosed and taught herein relates generally to topically-applied compositions for personal care and to the rejuvenation, renewal, repair and maintenance of healthy skin. More specifically, the invention disclosed herein is related to topically-applied skin care compositions for imparting rejuvenated skin exhibiting reduced wrinkles and an anti-aging effect.

2. Description of the Related Art

Human skin is often subjected to harsh external conditions, such as excessive sun exposure and general environmental exposures, which over time lead to the anatomic degradation of human skin. The symptoms of such degradation are most pronounced on the face and around the eyes, and often manifest themselves as wrinkles, blotchy discolorations, leathery skin texture, dryness, roughness, and premalignant growths. Though the skin has a natural ability to repair such damage, with aging, the ability of the skin to spontaneously repair itself decreases.

Consequently, there have long been efforts to treat human skin in an effort to slow or counter these aging effects on the skin, and renew or rejuvenate the dermal tissues.

Much of the early work in skin renewal and rejuvenation focused on the use of vitamin A as a therapeutic agent. Many of these early treatment methodologies involved the administration of vitamin A internally in the form of the ester of vitamin A, vitamin A palmitate. This approach changed over time to focus on the topical application of vitamin A in its acid form (retinoic acid), which in early reports suggested that vitamin A could potentially retard the effects of aging on the skin. However, due to the side effects of the use of retinoic acid in its topical form (painful and unpleasant peeling of the skin), the use of vitamin A in its various forms has limited applicability from a long-term treatment standpoint. Therefore, interest in a number of other natural products having known, potential skin rejuvenating effects has been the focus of much recent research for topical application.

It has been reported that application of select natural products may be associated with a reduced aging effect of the human skin. For example, U.S. Pat. No. 6,146,650 describes the use of liposomes to deliver collagen, avocado oil, aloe and vital nutrients such as Vitamins A, C, D and E to the skin. Also, U.S. Pat. No. 6,281,236 describes cosmetic compositions containing allantoin and an emulsifier such as natural beeswax for the treatment of skin.

Certain skin treatments target damaged tissue. Thus, U.S. Pat. No. 6,319,942, describes the use of alkanolamines such as dimethylaminoethanol (DMAE) for the treatment of scars; U.S. Pat. No. 6,296,861, describes the use of conjugated linoleic acid and fatty acid esters of vitamin C for treatment of skin damage; U.S. Pat. No. 6,191,121, describes the use of polyenoylphosphatidyl choline to treat skin damage; U.S. Pat. Nos. 5,965,618 and 5,709,868, describe the treatment of scar tissue using lipoic acid, and additionally, alpha-hydroxy acids, fatty acid esters of vitamin C, and tocopherol (vitamin E); and U.S. Pat. Nos. 5,554,647 and 5,643,586 describe the use of catecholamine or acetylcholine precursors for treatment of skin damage. Vitamins have been used to prevent or reverse skin damage, and in particular, skin damage associated with inflammation due to UV radiation. For example, U.S. Pat. Nos. 5,574,063, 5,545,398, 5,409,693, and 5,376,361 describe the use of fatty acid esters of ascorbic acid (e.g., vitamin C palmitate) or tocotrienol (vitamin E) for treatment and prevention of skin damage.

Skin creams may comprise several ingredients, some of which are beneficial to the skin, and others that promote absorption of active ingredients into the skin. For example, U.S. Pat. No. 4,362,747 describes a cream pack formulation which comprises a mixture of the following components: (1) propylene glycol and polyoxyethylene; (2) monopalmitate and glyoxyldiureide; (3) alcohol, beeswax, sorbitan monopalmitate, and polyoxyethylene; (4) alcohol, dimethicone copolyol, glyceryl monosterarate/polyoxyethylene; and (5) stearate and zinc or titanium oxide. U.S. Pat. No. 5,391,373 describes a skin cream comprising sodium lactate, a micellar complex of plant extracts, vitamin B, and glycosphingolipids, a protein complex of serum proteins, animal proteins, and glycogen, a carbohydrate based complex of dextran, glycine and glucosamine, a long-chain fatty acid ester of retinol, a long-chain fatty acid ester of ascorbic acid and a short chain fatty acid ester of tocopherol.

Certain treatments are designed to target specific skin problems. For example, U.S. Pat. Nos. 5,958,397, 5,922,331, 5,817,621, 5,658,580, 5,362,488, 5,322,685, 5,254,331, 4,760,096, 4,297,374, 4,268,526, 4,087,555, and 4,007,266 all describe the formulation of skin creams which address specific aspects related to skin care. For example, U.S. Pat. No. 5,817,621 describes a skin cream comprising a lipid ointment, vitamin A, a salicylic acid, D-camphor, a biogenic GABAergic substance, a dopaminergic substance, M-cholionolyics, pancreatin, ascorbic acid, pantothenic acid calcium salt, and vitamin D₂ as a means to cause a high trophoprotective effect followed by a restoration of skin physiological functions.

U.S. Pat. No. 7,608,642 describes pharmaceutical compositions and methods for managing wound and skin care, in particular methods and compositions that employ compounds that can promote skin cell renewal, wound healing, proliferation of fibroblasts and/or keratinocytes, and the production of collagen. These compositions include as active ingredients giberellic acid compounds, a jasmonic acid compound, and a zeatin compound.

Still, none of the skin creams developed thus far have been effective enough to be entirely embraced by those suffering from damaged skin or wanting to deter aging of the skin. Although there are some treatments that are known to remedy specific skin conditions, there is a need for a simple all-in-one cosmetic treatment that increases skin firmness to thereby reduce aging and damage to the skin.

The invention disclosed and taught herein is directed to skin rejuvenation, minor wound repair, and wrinkle reduction compositions and formulations in the form of cosmetic creams, as well as methods for treating the skin of a mammal such as a human to facilitate the management, prevention, and treatment of one or more such skin conditions.

### BRIEF SUMMARY OF THE INVENTION

Cream or lotion compositions and formulations are described herein, for use in the therapeutic renewal and rejuvenation of the skin of a patient. Specific target components may act, alone or in synergistic combination, to increase the generation of stem, epidermal, or other cells in the skin; to activate or increase collagen synthesis in the skin; to activate or increase endogenous hyaluronic acid synthesis in the epidermis; to activate or increase the hydration of the skin, and to activate or increase the stem cell and fibroblast migration within the epidermis to sites of needed repair on the skin. The use of the skin care composition cream, lotion, or other dermal application compositions of the present invention may yield progress towards dramatically younger looking skin, rehydration and a decrease in signs of aging such as dryness, thin skin, deep wrinkles and dull appearances.

In accordance with one aspect of the present disclosure, a topical cosmetic, pharmaceutical, or dermatological composition is described, the topical cosmetic, pharmaceutical, or dermatological composition comprising an effective quantity of L-arginine; at least one dermatologically acceptable compound comprising divalent zinc ions; and one or more extracts of a Panax, Cimicifuga, and/or a Trifolium species in a cosmetic vehicle.

In accordance with further aspects of the present disclosure, a method for conditioning the skin of a mammal is described, the method comprising applying topically to the skin of a patient a composition comprising L-arginine; at least one dermatologically acceptable compound comprising divalent zinc ions; and one or more extracts of a Panax, Cimicifuga, and/or a Trifolium species in a cosmetic vehicle.

In embodiments the Panax species is selected from the group consisting of Panax Araliaceae, Panax bipinnatifidus, Panax ginseng, Panax japonicus, Panax quinquefolius, Panax trifolius, Panax vietnamensis, Panax wangianus, and Panax zingiberensis. In embodiments the Cimicifuga species is selected from the group consisting of C. racemosa, C. dahurica, C. foetida, and C. acerina. In embodiments, the extract is from the root of the plant. In embodiments, the vehicle further comprises one or more additional ingredients selected from the group consisting of an emulsifier, a thickener, a skin emollient, and a inorganic particulate material.

In embodiments, the composition including L-arginine, at least one dermatologically acceptable compound comprising divalent zinc ions; one or more extracts of a Panax species, one or more extracts from a Cimicifuga species, and/or one or more extracts from a Trifolium species is in a gel, paste, cream, lotion, emulsion, or ointment vehicle.

In embodiments, the topical cosmetic, pharmaceutical, or dermatological composition is described which comprises at least one anti-oxidant; L-arginine; at least one dermatologically acceptable compound comprising divalent zinc ions; one or more extracts of a Panax species; one or more extracts of a Cimicifuga species; one or more extracts of a Trifolium species; and vitamin B₅ or a derivative thereof, all of which are contained in a cosmetic vehicle. This composition may further comprise an epidermal skin cell activator, a collagen synthesis activator, a hyaluronic acid synthesis activator, a skin hydration activator, and a fibroblast migration activator, singly or in combination.

### BRIEF DESCRIPTION OF THE DRAWING

The following FIGURE forms part of the present specification and is included to further demonstrate certain aspects of the present invention. The invention may be better understood by reference to this FIGURE in combination with the detailed description of specific embodiments presented herein.

FIG. 1 illustrates a general Illustration of the anatomy of the skin epidermis.

While the invention disclosed herein is susceptible to various modifications and alternative forms, only a few specific embodiments have been shown by way of example in the drawings and are described in detail below. The FIGURE and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DEFINITIONS

The following definitions are provided in order to aid those skilled in the art in understanding the detailed description of the present invention.

The term “dermatologically-acceptable,” as used herein, means that the described or referenced compositions or components thereof are suitable for use in contact with mammalian skin tissue without undue toxicity, incompatibility, instability, allergic response, and the like.

The term “dermis” refers to the layer of skin that forms the foundation upon which the epidermis lies. The primary cellular components of the dermis are the dermal fibroblasts that exist in a sea of extracellular matrix within the dermis.

The term “epidermis” refers to the layer of skin over the dermis. The epidermis is a stratified squamous epithelium, composed primarily of keratinocytes. Keratinocytes within the epidermis are organized into four layers including the basal, spinous, granular, and cornified layers.

The term “hexoses” as used herein refers to any six-membered D or L saccharide. Such hexoses include allose, altrose, glucose, mannose, galose, idose, galactose and talose. The hexose utilized may also be a deoxy hexose.

The term “keratinous tissue,” as used herein, refers to keratin-containing layers disposed as the outermost protective covering of mammals (e.g., humans, dogs, cats, etc.) that includes, but is not limited to, skin, lips, hair, toenails, fingernails, cuticles, hooves, etc.

The term “pentose” refers to any five-membered D or L saccharide or sugar. Such pentoses include ribose, arabinose, xylose and lyxose. The pentose utilized may also be a deoxy pentose.

The term “promote faster cell turnover” refers to the movement of cells from the basement membrane region of the dermis through the layers of the epidermis until they enucleate and are sloughed from the surface of the skin. The rate of cellular turnover can be measured as described herein, for example, in the Examples.

The term “safe and effective amount” as used herein means an amount of a compound or composition sufficient to significantly induce a positive benefit, such as a positive appearance of the skin or a positive feel to the skin, but low enough to avoid serious side effects, i.e., to provide a reasonable benefit to risk ratio, within the scope of sound judgment of the skilled artisan.

The term “GRAS compound”, as used herein, refers to those compounds which are classified as Generally Recognized As Safe compounds as certified by the U.S. FDA.

The term “sagging” as used herein means the laxity, slackness, or the like condition of skin that occurs as a result of loss of, damage to, alterations to, and/or abnormalities in dermal elastin.

The term “skin” refers to the outer covering of an animal body; the outermost layer of skin is called the epidermis, the layer beneath the epidermis is called the dermis.

The term “skin stem cells”, as used herein, refers to those stem cells that reside in the basal layer of the epidermis and/or at the base of hair follicles.

The terms “smoothing” and “softening” as used herein mean altering the surface of the skin and/or keratinous tissue such that its tactile feel is improved. “Signs of skin aging” include, but are not limited to, all outwardly visible or tactilely perceptible manifestations as well as any other macro or micro effects due to skin aging. Such signs may be induced or caused by intrinsic factors or extrinsic factors, e.g., chronological aging and/or environmental damage. These signs may result from processes that include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular System (e.g., telangiectasia or spider vessels), and underlying tissues, especially those proximate to the skin.

The term “stem cells”, as used herein, means those mammalian cells which maintain and repair the tissues in which they are found, including epidermal stem cells.

The term “sugar residue” means any sugar available to one of skill in the art. For example, a sugar residue can be a hexose or a pentose.

The term “topical application”, as used herein, means to apply or spread the compositions of the present invention onto the surface of the skin tissue or within a wound.

### DETAILED DESCRIPTION

The FIGURE described above and the written description of specific structures and functions below are not presented to limit the scope of what Applicants have invented or the scope of the appended claims. Rather, the FIGURE and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a Singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Applicants have created compositions for promoting skin renewal and rejuvenation, increasing cell proliferation, for stimulating collagen and fibronectin production in the skin, and for wound repair/tissue healing in the case of minor wounds to the dermal tissue. These compositions can also be used to mitigate the effects of aging in all types and layers of skin tissues including the dermis, epidermis, keratinous tissues, mucosal tissues and the like. Such compositions are useful for treating minor wounds, stimulating tissue growth, enhancing skin thickness, and for mitigating the effects of aging on skin. The compositions of the present invention contain an effective amount of a compound from a Panax species of ginseng plant, a compound from a Cimicifuga species of plant (black cohosh), and/or a compound from a Trifolium species of plant, L-arginine, at least one compound comprising divalent zinc ions; as well as acceptable carriers and other ingredients. These compositions may also further include one or more of a genistein compound, an epidermal skin cell activator, a collagen synthesis activator, a hyaluronic acid synthesis activator, a skin hydration activator, and a fibroblast migration activator, singly or in combination, as appropriate, in therapeutically effective amounts.

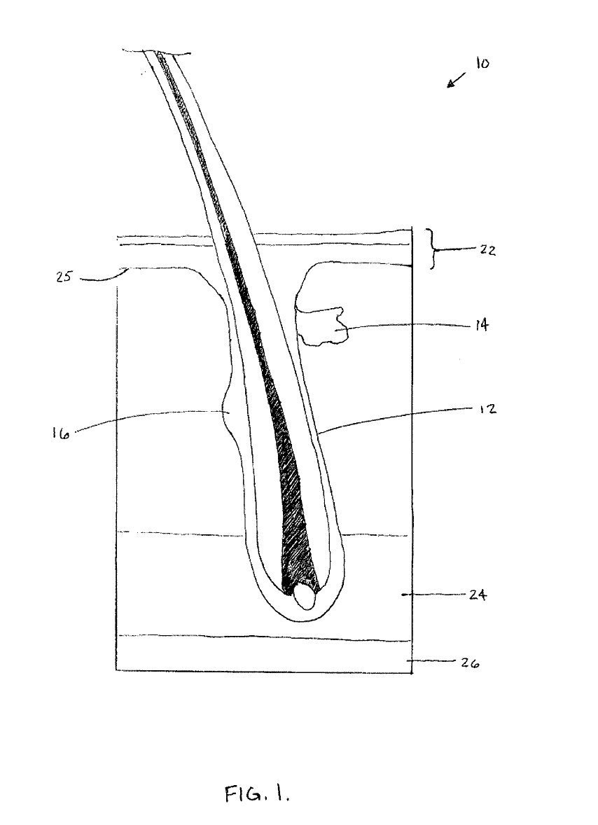

FIG. 1 is an Illustration of the general anatomy of a region of skin 10, shown in a perspective view illustrating the various subcutaneous layers 20 of the skin. Specifically, the skin is divided into the epidermis 22 (sometimes referred to as the intrafolicular epidermis), the dermis 24, and the hypodermis 26 (also known as the subcutaneous tissue, or pauniculus adiposus). The epidermis extends from about 0.05 mm to about 1.5 mm below the surface of the skin, and comprises the lamina lucinda and the lamina densa. The dermis 24 is below the epidermis, is separated from the epidermis by the dermoepidermal junction 25, and has a thickness from about 1.4 mm to about 4 mm, depending upon the location. The dermis comprises the upper, papillary dermis and the lower, reticular dermis (known as the “deep layer”). Below the dermis is the subcutaneous tissue 26, which has an average thickness of from about 1 cm to about 4 cm, although this thickness increases on obese individuals. As further illustrated in FIG. 1, a hair follicle 12 extends into the dermis layer 24. Surrounding the hair follicle 12 are typically located sebaceous glands 14, and bulge regions, 16, the latter of which has been associated with stem cell production in the skin of mammals.

Damage to regions of skin, 10, such as from minor wounds, or from the effects of aging or prolonged sun damage, can advantageously be counteracted with the compositions of the present disclosure. These compositions are skin rejuvenating, renewal, and/or wound treating compositions comprising an effective amount of a compound from a Panax species of ginseng plant, a compound from a Cimicifuga species of plant (black cohosh), and/or a compound from a Trifolium species of plant, L-arginine, at least one compound comprising divalent zinc ions, an optional genistein compound, and acceptable carriers and other ingredients suitable for use in preparing a therapeutic cosmetic formulation for topical application.

While not wishing to be limited by or bound to a specific theory, it is believed that the compositions described herein provide their therapeutic effect in combating skin wrinkles, in skin rejuvenation, and in minor wound repair, at least in part due to causing epidermal stem cell activation and stem cell motivated repair. In this sense, it has been found that the compositions of the instantly described compositions benefit by the synergistic effect of the inclusion of L-arginine, which acts to increase NO₂ processing and in turn increases the increased activity of epidermal stem cells.

In addition to the preferred active ingredients for the composition recited above, it is envisioned that alternative, equally acceptable compositions having the same therapeutic effects can be prepared, containing as additional or replacement active ingredients therapeutically effective amounts of extracts from chastetree berry (Vitex agnus-castus), dong quai (Angelica sinensis), evening primrose oil (Oenethera biennis), motherwort (Leonuruscardiaca), and licorice (Glycyrrhiza glabra), separately or in combination with the ingredients described herein.

Another particularly preferred additional active ingredient for the composition recited above is an extract from a Astralagus species, in particular from the species A. membranaceus, A. trojanus, A. zahlbruckneri, A. brachypterus, A. microcephalus, A. peregrines, A. caprinus, A. melanophrurius, A. oleifolius, a. trigonus, A. spinosus and A. verrucosus, and preferably from the root of those species. The solvent used for obtaining the extracts is preferably a polar organic solvent, optionally in admixture with water, such as aqueous lower alkanol solvent, e.g 70% or 95% ethyl alcohol. Such extracts are described in published US patent application US 2007/0122501 A1, which is incorporated herein by reference in its entirety.

Still another preferred additional active ingredient for the composition recited above is stem cells from plants. The term “stem cells of plants”, as used herein, means the following cells: 1. Callus or dedifferentiated somatic cells, and 2. plant stem cells from meristematic tissues. The former cells are more easily accessible and are usually in cosmetics. Plant species whose stem cells (callus) may be employed comprise certain cultivars of apple trees (e.g. “Uttwiler Spätlauber”), edelweiss and sea holly.

Ginsenosides

The topical compositions of the present invention preferably include one or more compounds contained within the extracts of the genus Panax, also known commonly as ginseng, such as extracts from the root of a Panax ginseng species, such as Panax Araliaceae, Panax bipinnatifidus, Panax ginseng, Panax japonicus, Panax quinquefolius, Panax trifolius, Panax vietnamensis, Panax wangianus, and Panax zingiberensis, as well as pseudoginseng species (e.g., Panax pseudoginseng). Specific compounds for use in the present formulations include extracts containing ginsenoside Rb1, Rb2, Rc, Rd, Re, Rf, Rg1, Ro, and combinations thereof, as well as any of these ginsenosides in their substantially pure form. The Panax compounds present in the compositions of the instant invention are in an amount ranging from about 0.01% to about 50%, or from about 0.1% to about 10%, or from about 0.5% to about 10%, or from about 1% to about 5%, or from about 2% to about 5%, by weight of the composition, of the Panax compound. The Panax compounds useful in the compositions of the present invention include compounds of general formula (I), below:

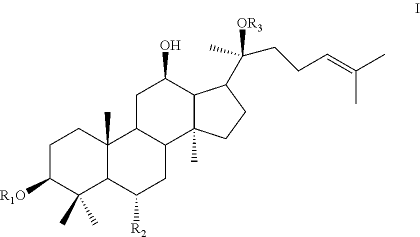

wherein R₁ is H or Glc-²Glc-; R₂ is H, Rha-²Glc-O—, Glc-²Glc-O—, Glc-O, O-Glc²-Rha, O-Glc²-Xyl, O-Glc²(Ac)-Xyl, or O-Glc²(Ac)-Rha; and R₃ is H, Glc-, Glc-⁶Glc-, Ara(p)-⁶Glc-, or Ara(f)-⁶Glc-, wherein “Glc”=β-D-glucopyranosyl or glucose; “Rha”=α-L-rhamnopyranosyl or rhamnose; “Ara(f)”=oc-L-arabinofuranosyl; “Ara(p)”=α-L-arabinopyranosyl; “Ara”=arabinose; and “Xyl”=β-D-xylopyranosyl, as well as derivatives and pharmaceutically acceptable salts thereof.

The Panax compounds may be included as the substantially pure material, e.g. pure Rb1, or as a mixture, or as an extract obtained by suitable physical and/or chemical Isolation from natural (e.g., plant) sources. Preferred extracts are obtained from the roots of the plants by means of a polar organic solvent optionally in admixture with water, such as aqueous ethanol.

Trifolium Extract

The compositions of the present invention may preferably include natural hormone extracts of clover and clover flowers, especially those of the genus Trifolium (e.g., Trifolium pratense or Trifolium subterraneum), such as flavonins, glycosides, isoflavones, saponins, soyasaponinsins, and extracts of the genus Trifolium containing combinations of such compounds, in amounts ranging from about 0.05 to about 20.0 wt. %, inclusive.

Exemplary extracts of the Trifolium species suitable for use with the compositions of the present invention will include one or more isoflavones of the following general structure (II), in an amount ranging from about 0.05 to about 20.0 wt. %, inclusive,

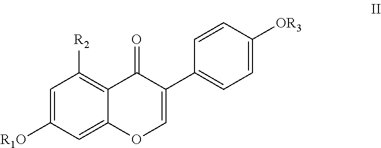

wherein R₁ is H or CH₃; R₂ is H, OH, CH₃ or OCH₃; and R₃ is H or CH₃, as well as derivatives and pharmaceutically acceptable salts thereof.

The Trifolium compounds may be included as the substantially pure material, or as an extract obtained by suitable physical and/or chemical Isolation from natural (e.g., plant) sources. Preferred extracts are obtained from the roots of the plant by means of a polar organic solvent optionally in admixture with water, such as aqueous ethanol.

Cimicifuga Compounds

The compositions of the present invention also preferably include one or more constituents of the Cimicifuga species of plants, including Cimicifuga racemosa, C. Simplex, C. rhizoma, C. dahurica, C. foetida, C. acerina, and C. heracleifolia, herein referred to generally as “cimicifuga compounds”, including the extracts of such Cimicifuga spp., particularly C. racemosa. The Cimicifuga compounds present in the compositions of the instant invention are in an amount ranging from about 0.01% to about 50%, or from about 0.1% to about 10%, or from about 0.5% to about 10%, or from about 1% to about 5%, or from about 2% to about 5%, by weight of the composition, of the Cimicifuga compound. The Cimicifuga compounds useful in the compositions of the present invention include compounds of formula (III), below,

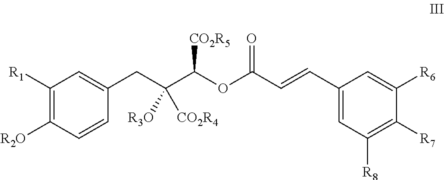

wherein R₁ H, OH, or OCH₃; R₂ is H, OH, or OCH₃; R₃ is H, CH₃, or CₙHₙ+₁; R₄ is H, Ac, CH₃, C₂H₅, Gal, or Glc; R₅ is H, Ac, CH₃, C₂H₅, Gal, or Glc; R₆ is H, OH, OCH₃, O-Gal, or O-Glc; R₇ is H, OH, OCH₃, O-Gal, or O-Glc; and R₈ is H, OH, OCH₃, O-Gal, or O-Glc, as well as derivatives thereof, and pharmaceutically acceptable salts thereof.

The Cimicifuga compounds may be included as the substantially pure material, or as an extract obtained by suitable physical and/or chemical Isolation from natural (e.g., plant) sources. Preferred extracts are obtained from the roots of the plant by means of a polar organic solvent optionally in admixture with water, such as aqueous ethanol.

Compound Comprising Divalent Zinc Ions

The compositions of the present invention contain at least one dermatologically acceptable compound comprising a divalent zinc ion. Plain zinc salts suitable for topical use are e.g. zinc sulfate, zinc gluconate, zinc acetate, zinc pyrithion, and zinc oxide. The zinc ion may also be complexed in a complex having a relatively low complex forming constant, e.g. Zn²⁺ complexes with ascorbic acid and amino acids, such glycine and lysine, as described in EP 1 064 946 B1, which is herein incorporated by reference in its entirety.

In embodiments, the at least one dermatologically acceptable compound comprising a divalent zinc ion is not plain particulate zinc oxide.

The zinc ion-containing compound may be present in the composition of the present invention in an amount ranging from about 0.0001% to about 15%, or from about 0.01% to about 10%, or from about 0.5% to about 10%, or from about 1% to about 5%, or from about 2% to about 5%, by weight of the composition, based on the amount of zinc in the compound.

In the following optional active components as well as components of the cosmetic vehicle are described.

Anti-Wrinkle Compounds

The compositions of the present invention may further contain a safe and effective amount of one or more chemical agents generally referred to herein as anti-wrinkle compounds. Exemplary anti-wrinkle compounds suitable for use in the compositions of the present invention include D- and L-amino acids, including the sulfur-containing D- and L-amino acids and their derivatives and salts, particularly the N-acetyl derivatives, a preferred example of which is N-acetyl-L-cysteine; thiols (e.g. ethane thiol); phytic acid, lipoic acid; lysophosphatidic acid, skin peel agents (e.g., phenol and the like), vitamin B₃ compounds and retinoids that enhance the health and/or appearance of skin tissues.

Vitamin B₃ Compounds

The compositions of the present invention may contain a safe and effective amount of a vitamin B₃ compound. When vitamin B₃ compounds are present in the compositions of the instant invention, the compositions can contain from about 0.01% to about 50%, or from about 0.1% to about 10%, or from about 0.5% to about 10%, or from about 1% to about 5%, or from about 2% to about 5%, by weight of the composition, of the vitamin B₃ compound.

As used herein, “vitamin B₃ compound” means a compound having the general formula IV, below:

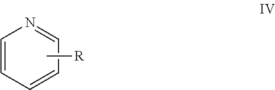

wherein R is —CONH₂ (e.g., niacinamide), —COOH (e.g., nicotinic acid) or —CH₂OH (e.g., nicotinyl alcohol), derivatives thereof, and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

The vitamin compounds may be included as the substantially pure material, or as an extract obtained by suitable physical and/or chemical Isolation from natural (e.g., plant) sources.

Examples of suitable vitamin B₃ compounds are well known in the art and are commercially available from a number of sources, including but not limited to, the Sigma Chemical Company (St. Louis, Mo.); ICN Biomedicals, Inc. (Irvin, Calif.) and Aldrich Chemical Company (Milwaukee, Wis.).

Vitamin B₅ Compounds

The compositions of the present invention may contain a safe and effective amount of a vitamin B₅ compound. When vitamin B₅ compounds are present in the compositions of the instant invention, the compositions can contain from about 0.01% to about 50%, or from about 0.1% to about 10%, or from about 0.5% to about 10%, or from about 1% to about 5%, or from about 2% to about 5%, by weight of the composition, of the vitamin B₅ compound.

As used herein, “vitamin B₅ compound” means a compound having the general formula V, below:

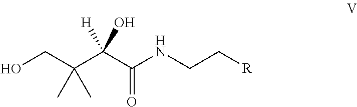

wherein R is OH, CO₂H, or S—S—(CH₂)₂NHC(O)CH₂(₂)—NHC(O)C(OH)CH(CH₃)₂CH₂OH(pantethine).

Vitamin C Compounds

The compositions of the present invention may contain a safe and effective amount of a vitamin C compound, or analog or derivative thereof.

Vitamin E Compounds

The compositions of the present invention may also contain vitamin E (tocopherol) and/or one or more Vitamin E compounds or derivatives of tocopherol. The tocopherols employed in the present invention include α-tocopherol, β-tocopherol, γ-tocopherol, and δ-tocopherol, as well as any of the known tocotrienols and combinations thereof, as well as derivatives of tocopherol of the structure VI, below:

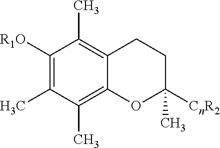

wherein n is an integer from 6 to 13, including 7, 8, 9, 10, 11, and 12; R₁ is hydrogen, alkyl, alkenyl, an ether, a silyl ether, or acetate; and R₂ is an optionally substituted nitrogen-containing heterocycle or a polycyclic nitrogen-containing heterocycle; and pharmaceutically acceptable salts thereof. Exemplary tocopherol derivatives suitable for use in the compositions described herein include, but are not limited to, (R)-2-(9-(1H-imidazol-1-yl)nonyl)-2,5,7,8-tetramethylchroman-6-ol; (R)-1-(9-(6-(tert-butyldimethylsilyloxy)-2,5,7,8-tetramethylchroman-2-Anonyl)-1H-1,2,3-triazole; (R)-1-(9-(6-(tert-butyldimethylsilyloxy)-2,5,7,8-tetramethylchroman-2-yl)-nonyl)-1H-1,2,4-triazole; (R)-2-(9-(1H-1,2,4-triazol-1-yl)nonyl)-2,5,7,8-tetramethyl-chroman-6-ol; (R)-2-(9-(1H-1,2,3-triazol-1-yl)nonyl)-2,5,7,8-tetramethylchroman-6-ol; (R)-2-(9-(6-(tert-butyldimethylsilyloxy)-2,5,7,8-tetramethylchroman-2-yl)-nonyl)-2H-1,2,3-triazoe; (R)-2-(9-(2H-1,2,3-triazol-2-yl)nonyl)-2,5,7,8-tetramethylchroman-6-ol; (R)-1-(9-(6-(tert-butyldimethylsilyloxy)-2,5,7,8-tetramethylchroman-2-yl)-nonyl)-1H-benzo[d]imidazole; (R)-2-(9-(1H-benzo[d]imidazol-1-yl)nonyl)-2,5,7,8-tetramethyl-chroman-6-ol; (R)-2-(9-(6-(tert-butyldimethylsilyloxy)-2,5,7,8-tetramethylchroman-2-yl-)nonyl)-2H-benzo[d][1,2,3]triazole; (R)-2-(9-(2H-benzo[d][1,2,3]triazol-2-yl)nonyl)-2,5,7,8-tetramethylchroman-6-ol; (R)-1-(9-(6-(tert-butyldimethylsilyloxy)-2,5,7,8-tetramethylchroman-2-yl)nonyl)-1H-benzo[d][1,2,3]triazole; (R)-2-(9-(1H-benzo[d][1,2,3]triazol-1-yl)nonyl)-2,5,7,8-tetramethylchroman-6-ol; 1-{9-[(R)-6-hydroxy-2,5,7,8-tetramethyl-chroman-2-yl]-nonyl}-5H-pyrimidine; and 1-{9-[(R)-6-hydroxy-2,5,7,8-tetramethyl-chroman-2-yl]-nonyl}-2H-pyrazine.

The vitamin E compounds present in the compositions of the instant invention are in an amount ranging from about 0.01% to about 20%, or from about 0.1% to about 10%, or from about 0.5% to about 10%, or from about 1% to about 5%, or from about 2% to about 5%, by weight of the composition, of the tocopherol, vitamin E compound.

Retinoids

The compositions of the present invention may also contain a retinoid. As used herein, “retinoid” includes all natural and/or synthetic analogs of Vitamin A or retinol-like compounds that possess the biological activity of Vitamin A in the skin as well as the geometric isomers and stereoisomers of these compounds. The retinoid can, for example, be retinol, retinol esters (e.g., 02-622 alkyl esters of retinol, including retinyl palmitate, retinyl acetate, retinyl propionate), retinal, and/or retinoic acid (including all-trans retinoic acid and/or 13-c/s-retinoic acid). In some embodiments, retinoids other than retinoic acid are used. These compounds are available in the art and are commercially available from a number of sources, e.g., Sigma Chemical Company (St. Louis, Mo.), and Boehringer Mannheim (Indianapolis, Ind.). Other retinoids that are useful in the therapeutic compositions described herein are described in U.S. Pat. No. 4,677,120; U.S. Pat. No. 4,885,311; U.S. Pat. No. 5,049,584; U.S. Pat. No. 5,124,356; and U.S. patent Reissue No. 34,075. Other suitable retinoids are tocopheryl-retinoate, tocopherol ester of cis- or trans-retinoic acid, adapalene (6-[3-(1-adamantyl)-4-methoxyphenyl]-2-naphthoic acid), and tazarotene (ethyl 6-[2-(4,4-dimethylthiochroman-6-yl)-ethynyl]nicotinate). Desirable retinoids include retinol, retinyl palmitate, retinyl acetate, retinyl propionate, retinal and combinations thereof.

The retinoid may be included as the substantially pure material, or as an extract obtained by suitable physical and/or chemical Isolation from natural (e.g., plant) sources. In some embodiments, the retinoid is substantially pure (>95% pure), or essentially pure (>98% pure).

The compositions of this invention may contain a safe and effective amount of the retinoid, such that the resultant composition is safe and effective for regulating or improving the condition of skin tissues. The compositions and methods of the invention can improve visible and/or tactile discontinuities in skin, or improve signs of skin aging. The compositions preferably contain from about 0.005% to about 2% by weight, or from about 0.01% to about 2% by weight, retinoid. Retinol can also be used in an amount of from about 0.01% to about 1.5% weight. Retinol esters can be used in an amount of from or about 0.01% by weight to or about 2.5% by weight (e.g., about 1%). Retinoic acids can be used in an amount of from or about 0.01% by weight to or about 2.5% weight. Tocopheryl-retinoate, adapalene, and tazarotene can be used in an amount of from or about 0.01% to or about 2% weight.

Amino Acids

Amino acids, including but not limited to alpha-amino acids, beta-amino acids, and derivatives and analogs thereof, in both the L- and D-form (as appropriate) may be included in the compositions of the present invention in safe and effective amounts. As used herein, the term “amino acid” refers to both naturally-occurring and synthetic amino acids. Exemplary amino acids suitable for use in the compositions described herein include but are not limited to L-arginine, L-aspartamine, aspartic acid, L-proline, L-serine, L-tyrosine, L-tryptophan, L-lysine, L-glycine, L-leucine, L-alanine, L-phenylalanine, L-valine, L-cysteine, L-methionine, and L-glutamine. The compositions of the present invention include at least the amino acid L-arginine, in a therapeutically effective amount ranging from about 0.1 to about 15.0 wt. %.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides, as well as peptidomimetics. Also useful herein are naturally occurring and commercially available compositions that contain peptides.

Suitable dipeptides for use herein include Carnosine (beta-ala-his). Suitable tripeptides for use herein include, gly-his-lys, arg-lys-arg, and his-gly-gly. Preferred tripeptides and derivatives thereof include palmitoyl-gly-his-lys, which may be purchased as Biopeptide CL™ (100 ppm of palmitoyl-gly-his-lys commercially available from Sedenna, France); Peptide CK (arg-lys-arg); Peptide CK+ (ac-arg-lys-arg-NH₂); and a copper derivative of his-gly-gly sold commercially as lamin, from Sigma (St. Louis, Mo.).

When included in the present compositions, peptides can be present in amounts of from about 1×10″⁶% to about 10%, or from about 1×10″⁶% to about 0.1%, or from about 1×10″⁶% to about 0.01%, by weight of the composition. In certain compositions, where the peptide is a specifically desired peptide (such as the Arg-Gly-Asp tripeptide), the compositions can contain from about 0.1% to about 5%, by weight of the composition, of such peptides. In other embodiments wherein the peptide-containing compositions, Matrixyl™, and/or Biopeptide CL™ are included, the compositions can contain from about 0.1% to about 10%, by weight compositions, of Matrixyl™ and/or Biopeptide CL™ peptide-containing compositions.

Anti-Oxidant/Radical Scavenger

The compositions of the present invention may include a safe and effective amount of an anti-oxidant/radical scavenger. The anti-oxidant/radical scavenger is especially useful for providing protection against ultraviolet radiation that can cause increased scaling or texture changes in the stratum corneum and against other environmental agents that can cause skin damage.

A safe and effective amount of an anti-oxidant/radical scavenger may be added to the compositions of the subject invention, for example, from about 0.1% to about 10%, or from about 1% to about 5%, by weight of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox™), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lysine, pidolate, arginine pidolate, nordihydroguaiaretic acid, biofiavonoids, curcumin, lysine, methionine, proline, Superoxide dismutase, silymarin, tea extracts, grape skin/seed extracts, melanin, and rosemary extracts may be used. Other anti-oxidants/radical scavengers are selected from tocopherol sorbate and other esters of tocopherol. For example, the use of tocopherol sorbate in topical compositions and applicable to the present invention is described in U.S. Pat. No. 4,847,071.

Chelators

The compositions of the present invention may also contain a safe and effective amount of a or chelating agent. As used herein, “chelator” or “chelating agent” means an active agent capable of removing a metal ion from a system by forming a complex so that the metal ion cannot readily participate in or catalyze chemical reactions. The inclusion of a chelating agent is useful for providing protection against ultraviolet radiation that can contribute to excessive scaling or skin texture changes and against other environmental agents that can cause skin damage.

A safe and effective amount of a chelating agent may be added to the compositions of the subject invention, for example, from about 0.1% to about 10%, or from about 1% to about 5%, of the composition. Exemplary chelators that are useful herein are disclosed in U.S. Pat. No. 5,487,884; International Publication No. 91/16035, Bush et al., published Oct. 31, 1995; and International Publication No. 91/16034, Bush et al., published Oct. 31, 1995. In some embodiments, the chelators used in compositions of the subject invention include, for example, furildioxime, furilnonoxime, and derivatives thereof.

Flavonoids

The compositions of the present invention may optionally contain a flavonoid compound. Flavonoids are broadly disclosed in U.S. Pat. Nos. 5,686,082 and 5,686,367, both of which are herein incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, CᵣC₈ alkyl, CᵣC₄ alkoxyl, O-glycoside, and the like or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (e.g. unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxycoumarin, 7-hydroxycoumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

In some embodiments, unsubstituted flavanone, methoxy flavanones, unsubstituted chalcone, 2′,4-dihydroxy chalcone, and mixtures thereof are used in the compositions of the invention. In other embodiments, unsubstituted flavanone, unsubstituted chalcone (especially the trans isomer), and mixtures thereof are used in the compositions of the invention.

Flavonoids can be synthesized or obtained as extracts from natural sources (e.g., plants). The naturally sourced material can also further be derivatized (e.g., an ester or ether derivative prepared following extraction from a natural source). Flavonoid compounds useful herein are also commercially available from a number of sources, e.g., Indofine Chemical Company, Inc. (Somerville, N.J.), Steraloids, Inc. (Wilton, N.H.), and Aldrich Chemical Company, Inc. (Milwaukee, Wis.). Mixtures of such flavonoid compounds may also be used.

The flavonoid compounds can be present in the invention, for example, at concentrations of from about 0.01% to about 20%, more preferably from about 0.1% to about 10%, and still more preferably from about 0.5% to about 5%.

Anti-Inflammatory Agents

A safe and effective amount of an anti-inflammatory agent may be added to the compositions of the present invention, for example, from about 0.1% to about 10%, or from about 0.5% to about 5%, of the composition may include an anti-inflammatory agent, including non-steroidal anti-inflammatory agents (NSAIDS). The anti-inflammatory agent can enhance the appearance of the skin, for example, by contributing to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenol one, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. In some embodiments, the steroidal anti-inflammatory used is hydrocortisone.

A second class of anti-inflammatory agents that is useful in the compositions includes the nonsteroidal anti-inflammatory agents. A variety of compounds are encompassed by this group. For detailed disclosure of the chemical structure, synthesis, side effects, etc. of non-steroidal anti-inflammatory agents, one may refer to Standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York.

Specific non-steroidal anti-inflammatory agents useful in the composition invention include, but are not limited to: 1) the oxicams, such as piroxicam, isoxicam, tenoxicam, sudoxicam, and CP-14,304; 2) benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is useful for topical application. Of the nonsteroidal anti-inflammatory agents, ibuprofen, naproxen, flufenamic acid, etofenamate, aspirin, mefenamic acid, meclofenamic acid, piroxicam and felbinac are often used. Ibuprofen, naproxen, ketoprofen, etofenamate, aspirin and flufenamic acid are frequently used.

Moreover, so-called “natural” anti-inflammatory agents are useful in methods of the present invention. Such agents may suitably be obtained as an extract by suitable physical and/or chemical Isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, or C₁₀-C₂₄, or C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Anti-Cellulite Agents

The compositions of the present invention may also contain a safe and effective amount of an anti-cellulite agent. Suitable agents may include, but are not limited to, xanthine compounds (e.g., caffeine, theophylline, theobromine, and aminophylline).

Topical Anesthetics

The compositions of the present invention may also contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Tanning Compounds

The compositions of the present invention may contain a tanning compound. When present, the compositions can contain from about 0.1% to about 20%, or from about 2% to about 7%, or from about 3% to about 6%, by weight of the composition, of dihydroxyacetone as an artificial tanning compound.

Dihydroxyacetone, which is also known as DMA or 1,3-dihydroxy-2-propanone, is a white to off-white, crystalline powder. This material can be represented by the chemical formula C₃H₆O₃. The compound can exist as a mixture of monomers and dimers, with the dimers predominating in the solid crystalline state. Upon heating or melting, the dimers break down to yield the monomers. This conversion of the dimeric form to the monomeric form also occurs in aqueous solution. Dihydroxyacetone is also known to be more stable at acidic pH values. See The Merck Index, Tenth Edition, entry 3167, p. 463 (1983), and “Dihydroxyacetone for Cosmetics”, E. Merck Technical Bulletin, 03-304 110.

Skin-Lightening Agents

The compositions of the present invention may optionally contain one or more skin-lightening agents. When used, the compositions can contain from about 0.1% to about 10%, or from about 0.2% to about 5%, or from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl Phosphate), and extracts (e.g., mulberry extract, placental extract). Skin lightening agents suit able for use herein also include those described in the PCT publication No. 95/34280, in the name of Hillebrand, corresponding to PCT application Ser. No. U.S. 95/07432, filed Jun. 12, 1995; and co-pending U.S. application Ser. No. 08/390,152 filed in the names of Kvalnes, Mitchell A. DeLong, Barton J. Bradbury, Curtis B. Motley, and John D. Carter, corresponding to PCT Publication No. 95/23780, published Sep. 8, 1995.

Skin Soothing and Skin Healing Compounds

The compositions of the present invention may comprise a skin soothing or skin-healing compound. Skin soothing or skin healing compounds suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing compound may be added to the present composition, foe example, from about 0.1% to about 30%, or from about 0.5% to about 20%, or from about 0.5% to about 10%, by weight of the composition formed.

Anti-Microbial or Anti-Fungal Compounds

The compositions of the present invention may contain an anti-microbial or anti-fungal compound. Such compounds are capable of destroying microbes, preventing the development of microbes or preventing the pathogenic action of microbes. A safe and effective amount of an anti-microbial or anti-fungal compound may be added to the present compositions, for example, from about 0.001% to about 10%, or from about 0.01% to about 5%, or from about 0.05% to about 2%.

Examples of antimicrobial and antifungal compounds suitable for use with the presently described compositions include but are not limited to 13-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netihnicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate; kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Examples of compounds useful herein include those selected from benzoyl peroxide, 3-hydroxy benzoic acid, glycolic acid, lactic acid, 4-hydroxy benzoic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4^{I}-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

Sunscreen Compounds

Exposure to ultraviolet light can result in excessive scaling and texture changes of the stratum corneum. Therefore, the compositions of the subject invention may optionally contain a sunscreen compound. As used herein, “sunscreen compound” includes both sunscreen agents and physical sun blocks. Suitable sunscreen compounds may be organic or inorganic, and preferably are GRAS compounds.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens can be present in the amount of from about 0.1% to about 20%, or from about 0.5% to about 10%, or from about 1% to about 5%, by weight of the composition.

A wide variety of conventional organic sunscreen compounds are suitable for use herein. In ‘The Handbook of Cosmetic Science and Technology, 3^{rd} Edition” [Marc Paye, H. I. Maibach, & A. O. Barel, eds.; 2009], there are disclosed numerous suitable compounds for use as sunscreen compounds in the compositions of the present disclosure, including but not limited to: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylate esters (amyl, phenyl, octyl, benzyl, menthyl, glyceryl, and di-pro-pyleneglycol esters); cinnamic acid derivatives (menthyl and benzyl esters, α-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy-or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

Desirable compounds include 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and mixtures of these compounds.

In some embodiments, the organic sunscreen compounds used in the compositions of the invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene and mixtures thereof.

Useful sunscreen compounds are also described in U.S. Pat. No. 4,937,370, and U.S. Pat. No. 4,999,186. The sun-screening agents disclosed therein have, in a single molecule, two distinct chromophore moieties that exhibit different ultra-violet radiation absorption spectra. One of the chromophore moieties absorbs predominantly in the UVB radiation range and the other absorbs strongly in the UVA radiation range.

Desirable members of this class of sun-screening agents are 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoyl-methane; N,N-di-(2-ethylhexyl)-4-aminobenzoic acid ester of 2-hydroxy-4-(2-hydroxyethoxy)-benzophenone; and N,N-di-(2-ethylhexyl)-4-aminobenzoic acid ester of 4-(2-hydroxyethoxy)-dibenzoylmethane and mixtures thereof. Other desirable sunscreen compounds include 4,4′-t-butylmethoxydibenzoyl-methane, 2-ethylhexyl-p-methoxycinnamate, phenyl benzimidazole sulfonic acid, and octocrylene.

A safe and effective amount of the organic sunscreen compound is used, typically from about 1% to about 20%, more typically from about 2% to about 10% by weight of the composition. Exact amounts will vary depending upon the sunscreen or sunscreens chosen and the desired Sun Protection Factor (SPF).

Particulate Material

The compositions of the invention may contain a particulate material, for example, a inorganic, metallic oxide. These particulates can be coated or uncoated, charged or uncharged. Charged particulate materials are disclosed in U.S. Pat. No. 5,997,887, to Ha, et al., incorporated herein by reference. Particulate materials useful herein include but are not limited to bismuth oxychloride, iron oxide, mica, mica treated with barium sulfate and TiO₂, silica, nylon, polyethylene, talc, styrene, polypropylene, ethylene/acrylic acid copolymer, sericite, titanium dioxide, bismuth oxychloride, iron oxide, aluminum oxide, silicone resin, barium sulfate, calcium carbonate, cellulose acetate, polymethyl methacrylate, and mixtures thereof.

Inorganic particulate materials suitable for use herein also include metal oxides wherein the metals are from the transition metal series of the Periodic Table of Elements, including but not limited to TiO₂, ZnO, or ZrO₂, all of which are commercially available from a number of sources. One example of a suitable particulate material contains the material available from U.S. Cosmetics (TRONOX TiO₂ series, SAT-T CR₈37, a rutile TiO₂). Particulate materials can be present in the composition in levels of from about 0.01% to about 2%, or from about 0.05% to about 1.5%, or from about 0.1% to about 1%, by weight of the composition.

Conditioning Agent

The compositions of the present invention may contain a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose, glucosamine); hyaluronic acid; lactamide monoethanolamine; acetamide monoethanola mine; panthenol; allantoin; and mixtures thereof. Also useful herein are the propoxylated glycerols described in U.S. Pat. No. 4,976,953, to Orr et al.

Also useful are various C₁-C₃₀ monoesters and polyesters of sugars and related materials. These esters are derived from a sugar or polyol moiety and one or more carboxylic acid moieties. Such ester materials are further described in, U.S. Pat. No. 2,831,854; U.S. Pat. No. 4,005,196; U.S. Pat. No. 4,005,195; U.S. Pat. No. 5,306,516; U.S. Pat. No. 5,306,515; U.S. Pat. No. 5,305,514; U.S. Pat. No. 4,797,300; U.S. Pat. No. 3,963,699; U.S. Pat. No. 4,518,772; and U.S. Pat. No. 4,517,360.

Desirable conditioning agents are selected from urea, guanidine, sucrose polyester, panthenol, dexpanthenol, allantoin, and combinations thereof.

Cosmetic Vehicle—Product Forms

The present compositions may present themselves in a wide variety of product forms, including, for example, lotions, creams, gels, ointments, sticks, sprays, or pastes. These product forms may comprise several types of carriers, including, but not limited to, solutions, aerosols, emulsions, gels, solids, and liposomes. The carrier is frequently formulated as an emulsion, as described further below.

When the composition is formulated as an ointment, it may comprise a simple carrier base of animal or vegetable oils or semi-solid hydrocarbons, or an absorption ointment base which absorbs water to form an emulsion. Aerosols can be formed by adding a propellant, such as halogenated hydrocarbons known in the art, to a solution of the subject composition in a carrier such as described above. Aerosols are typically applied to the skin as a spray-on product.

The compositions of the present invention comprise a dermatologically acceptable carrier, within which the active ingredient and other components are incorporated, to allow these components to be delivered to the skin at an appropriate concentration. The carrier may contain one or more dermatologically acceptable solid, semi-solid or liquid fillers, diluents, solvents, extenders and the like. The carrier may be solid, semi-solid or liquid; preferred carriers are substantially liquid. The carrier should be physically and chemically compatible with the active ingredient and other components described herein. Preferred carriers contain a dermatologically acceptable, hydrophilic diluent; e.g., water, lower monovalent alcohols, low molecular weight glycols and polyols, such as propylene glycol, polyethylene glycol, polypropylene glycol, glycerol, butylene glycol, 1,2,4-butanetriol, sorbitol esters, 1,2,6-hexanetriol, and butanediol, ethoxylated ethers, propoxylated ethers, and combinations thereof. Water is a preferred diluent. The composition preferably comprises from about 60% to about 99% of the hydrophilic diluent.

In one embodiment, the formulation comprises an emulsion containing a hydrophilic phase, e.g., water or other hydrophilic diluent, and a hydrophobic phase, e.g., a lipid, oil or oily material, where one phase is dispersed in the other, continuous, phase. Examples are oil-in-water emulsions, water in oil emulsions, and water-in-silicone emulsions. Generally, the emulsion contains about 1% to 98% of the hydrophilic phase and about 1% to 50% of the hydrophobic phase. The emulsion may also comprise a gel network or a multiphase emulsion.

Preferred emulsions have an apparent viscosity at room temperature of from about 5,000 to about 200,000 centipoise (cps), depending on the physical form of the formulation. For example, a lotion may have an apparent viscosity of from about 10,000 to about 40,000 cps, and a cream may have an apparent viscosity of from about 60,000 to about 160,000 cps.

Suitable hydrophobic components employed in emulsions include, for example, vegetable oils, e.g. safflower oil, coconut oil, cottonseed oil, palm oil, soybean oil, and the like, which may be hydrogenated; animal fats and oils, such as lanolin; mineral oil; petrolatum, or petroleum jelly; or C7 to C40 hydrocarbons, e.g. such as dodecane, squalane, cholestanes, hydrogenated polyisobutylene, docosane, and various isoparaffins (branched hydrocarbons). Also suitable are esters of C1-C30 carboxylic acids and of C2-C30 dicarboxylic acids, where the alcohol component is derived from C1-C30 alcohols, glycols, or glycerols. Examples include, but are by no means limited to, isopropyl myristate, methyl palmitate, myristyl propionate, cetyl palmitate, dioctyl maleate, dioctyl sebacate, caprylic/capric triglyceride, PEG-8 caprylic/capric triglyceride, and ethylene glycol distearate; as well as propoxylated and ethoxylated derivatives thereof.

C1-C30 mono- and polyesters of sugars or other polyol moieties may also be used, as is known in the art, and include, for example, liquid materials such as glucose tetraoleate, glucose and mannose tetraesters of soybean oil fatty acids, galactose tetraesters of oleic acid, sorbitol hexaesters of unsaturated soybean oil fatty acids, and sucrose octaoleate. Solid materials include, for example, a sucrose polyester in which the degree of esterification is 7-8, and in which the fatty acid moieties are C18 mono- and/or di-unsaturated and behenic. Esters suitable for use in cosmetic emulsions are further described in, for example, U.S. Pat. Nos. 4,005,196, 5,306,516, 4,797,300, and 4,518,772. Also useful are C4-C20 alkyl ethers of polypropylene glycols, e.g. PPG-14 butyl ether, PPG-15 stearyl ether, dioctyl ether, dodecyl octyl ether, and mixtures thereof.

The hydrophobic component employed in an emulsion may also be an organopolysiloxane oil, such as disclosed in U.S. Pat. No. 5,069,897 (Orr). Examples of suitable organopolysiloxane oils include polyalkylsiloxanes, cyclic polyalkylsiloxanes, and polyalkylarylsiloxanes. Commercially available polyalkylsiloxanes include the polydimethylsiloxanes, which are also known as dimethicones, examples of which include the Vicasil™ series (General Electric) and the Dow Corning™ 200 series. Examples of alkyl-substituted dimethicones include cetyl dimethicone and lauryl dimethicone. Commercially available cyclic polyalkylsiloxanes include the cyclomethicones. Also useful are materials such as trimethylsiloxysilicate, such as that sold as a mixture with dimethicone as Dow Corning™ 593 fluid, and polyalkylaryl siloxanes.

Components

Formulations of the present invention, particularly emulsions, preferably include one or more components selected from emulsifiers, surfactants, structuring agents, thickeners, and emollients, as described below.

Emulsifiers and Surfactants

An emulsifier and/or surfactant is employed to disperse and suspend the discontinuous phase within the continuous phase. The surfactant should be hydrophilic enough to disperse in the hydrophilic phase; preferred surfactants are those having an HLB of at least about 8. The choice of surfactant will also depend upon the pH of the composition and the other components present.

Preferred hydrophilic surfactants are selected from nonionic surfactants, including those broadly defined as condensation products of long chain alcohols, e.g. C₈₋₃₀ alcohols, with sugar or starch polymers, i.e., glycosides. Commercially available examples include decyl polyglucoside (available as APG 325 CS from Henkel) and lauryl polyglucoside (available as APG 600 CS and 625 CS from Henkel). Other useful nonionic surfactants include alkylene oxide esters and diesters of fatty acids, and alkylene oxide ethers of fatty alcohols, as well as the condensation products of alkylene oxides with both fatty acids and fatty alcohols. Nonlimiting examples of alkylene oxide-derived nonionic surfactants include ceteth-12, ceteareth-10, steareth-12, PEG-10 stearate, PEG-100 stearate, PEG-20 glyceryl stearate, PEG-80 glyceryl tallowate, PEG-30 glyceryl cocoate, PEG-200 glyceryl tallowate, PEG-8 dilaurate, PEG-10 distearate, and mixtures thereof. Still other useful nonionic surfactants include polyhydroxy fatty acid amides, such as coconut alkyl N-methyl glucoside amide.

The hydrophilic surfactants useful herein can also include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; or U.S. Pat. No. 5,011,681 (Ciotti et al.). Cationic surfactants include, for example, cationic ammonium salts, such as quaternary ammonium salts, and amino-amides. Anionic surfactants include the alkoyl isethionates (e.g., C12-C30), alkyl and alkyl ether sulfates and phosphates, alkyl methyl taurates, and alkali metal salts of fatty acids. Examples of amphoteric and zwitterionic surfactants include derivatives of aliphatic secondary and tertiary amines in which one aliphatic substituent contains from about 8 to about 22 carbon atoms and one contains an anionic water solubilizing group, e.g., carboxy, sulfonate, sulfate, phosphate, or phosphonate. Examples are alkyl imino acetates, iminodialkanoates and aminoalkanoates, imidazolinium and ammonium derivatives. Other suitable amphoteric and zwitterionic surfactants include betaines, sultaines, hydroxysuitaines, alkyl sarcosinates (e.g., C12-C30), and alkanoyl sarcosinates.

Silicone containing emulsifiers or surfactants include dimethicone copolyols, i.e. polydimethyl siloxanes having polyether side chains, as well as dimethicone copolyols modified with pendant alkyl, cationic, anionic, amphoteric, and zwitterionic moieties. Dimethicone copolyol emulsifiers useful herein are described, for example, in U.S. Pat. No. 4,960,764 (Figueroa, Jr. et al.); G. H. Dahms et al., Cosmetics & Toiletries, vol. 110, pp. 91-100, 1995; M. E. Carlotti et al., J. Dispersion Science & Technology, 13(3), 315-336 (1992); P. Hameyer, HAPPI 28(4), pp. 88-128 (1991); J. Smid-Korbar et al., Intl Journal of Cosmetic Science, 12, 135-139 (1990); and D. G. Krzysik et al., Drug and Cosmetic Industry, vol. 146(4) pp. 28-81 (1990).

Structuring Agents

The present formulations, particular when in the form of an oil-in-water emulsion, may contain a structuring agent, preferably at a level of about 2% to about 9%. Preferred structuring agents are those having an HLB (hydrophile-lipophile balance) of about 1-8 and a melting point of at least about 45° C. Suitable structuring agents include, for example, saturated C14 to C30 fatty alcohols or amines, which may contain 1 to about 5 moles of ethylene oxide; saturated C16 to C30 diols; saturated C16 to C30 monoglycerol ethers; C14 to C30 saturated fatty acids, which may be hydroxylated or ethoxylated; C14 to C30 saturated glyceryl monoesters having a monoglyceride content of about 40% or more; C14 to C30 saturated polyglycerol esters having from about 1 to about 3 alkyl groups and from about 2 to about 3 saturated glycerol units; C14 to C30 glyceryl monoethers; C14 to C30 sorbitan mono/diesters or saturated methyl glucoside esters, which may be ethoxylated and/or contain 1 to about 5 moles of ethylene oxide; C14 to C30 saturated sucrose mono/diesters; C14 to C30 saturated polyglucosides having an average of 1 to 2 glucose units, and mixtures thereof. In selected embodiments, the structuring agent includes stearyl alcohol, cetyl alcohol, behenyl alcohol, a polyethylene glycol ether of stearyl or cetyl alcohol having an average of about 2 ethylene oxide units, and mixtures thereof.

Thickening Agents

The compositions of the present invention can contain one or more thickening agents, in an amount from about 0.1% to about 5%, or from about 0.1% to about 4%, or from about 0.25% to about 3%, by weight of the composition. Non-limiting classes of thickening agents include those selected from the following:

a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the CARBOPOL™ 900 series from B.F. Goodrich (e.g., CARBOPOL™). In addition, other suitable carboxylic acid polymeric agents include copolymers of Ci₀-so alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/Ci₀-so alkyl acrylate crosspolymers and are commercially available as Carbopol™ 1342, Carbopol™ 1382, Pemulen TR-1, and Pemulen TR-2, from B.F. Goodrich. In other words, examples of carboxylic acid polymer thickeners useful herein are those selected from carbomers, acrylates/Cio-Cao alkyl acrylate crosspolymers, and mixtures thereof.

b) Crosslinked Polyacrylate Polymers

The compositions of the present invention can optionally contain crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. No. 5,100,660, to Hawe et al.; U.S. Pat. No. 4,849,484, to Heard; U.S. Pat. No. 4,835,206, to Farrar et al.; U.S. Pat. No. 4,628,078 to Glover et al.; U.S. Pat. No. 4,599,379 to Fiesher et al.; and EP 228,868, to Farrar et al.

c) Polyacrylamide Polymers

The compositions of the present invention can optionally contain polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Trade name Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

d) Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. In these polymers, the hydroxy groups of the cellulose polymer is hydroxyalkylated (preferably hydroxyethylated or hydroxypropylated) to form a hydroxyalkylated cellulose that is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the trade name Natrosol™ CS Plus from Aqualon Corporation (Wilmington, Del.).

Other useful polysaccharides include scleroglucans that are a linear chain of (1-3) linked glucose units with a (1-6) linked glucose every three units, a commercially available example of which is CLEAROGEL™ CS11 from Michel Mercier Products Inc. (Mountainside, N.J.).

e) Gums

Other thickening and gelling agents useful herein include materials that are primarily derived from natural sources. Nonlimiting examples of these gelling agent gums include acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Compositions of the invention can therefore include desirable thickening agents such as carboxylic acid polymers, crosslinked polyacrylate polymers, polyacrylamide polymers, and mixtures thereof, more preferably selected from carboxylic acid polymers, polyacrylamide polymers, and mixtures thereof.

Emollients

The present formulations may also include a dermatologically acceptable emollient, e.g. at a level of about 2% to about 50%, depending on the physical form of the formulation. For example, lotions typically comprise about 5% to 10% emollient and about 60% to 80% water. A cream typically comprises about 10% to 20% emollient and about 50% to 75% water. An ointment may comprise about 2% to 10% emollient and about 0.1% to 2% of a thickening agent as described below. Generally, the emollient is present at a level of about 5% to about 25%.

Emollients are typically water-immiscible, oily or waxy materials which serve to lubricate the skin. An emollient may be selected from one or more of the following classes: triglyceride esters, which include, for example, vegetable and animal fats and oils; acetylated or ethoxylated glycerides; alkyl or alkyenyl esters of fatty acids, e.g. methyl palmitate, isopropyl isostearate, diisohexyl adipate, cetyl lactate, oleyl stearate, and the like; long chain fatty acids or alcohols such as myristic, palmitic, stearic, oleic, behenic, hydroxystearyl, and the like; lanolin and lanolin derivatives; polyhydric alcohol esters, e.g. mono and di-fatty acid esters of ethylene glycol, diethylene glycol, polyethylene glycol (200-6000), propylene glycol, and polypropylene glycol, and sorbitan, which may be ethoxylated; wax esters such as beeswax, spermaceti, and ethoxylated derivatives thereof; vegetable waxes such as carnauba and candelilla waxes; phospholipids such as lecithin and derivatives thereof, sterols, such as cholesterol and its fatty acid esters; and fatty acid amides.

Additional types of conditioning compounds include polyhydric alcohols and their derivatives, such as, for example, polypropylene glycol, hydroxypropyl sorbitol, pentaerythritol, xylitol, ethoxylated glycerol, soluble collagen, dibutyl phthalate, or gelatin. Also useful are ammonium and quaternary alkyl ammonium glycolates and lactates; aloe vera gel; and hyaluronic acid and derivatives thereof.

Inorganic Agents

Inorganic agents with or without organic modifications are usually part of the composition. The total amount of these agents in the composition is in the range from about 0.5 to about 5% by weight of the composition. The inorganic agent preferably comprises at least one smectite clay. Preferably, at least 20% by weight of the inorganic agent is a smectite clay, more preferably at least 30%, even more preferably at least 40% and optimally at least 50%.

Preferably the smectite clay is chosen from the group consisting of: aluminum silicates, such as the montmorillonites (bentonites, hectorites and derivatives thereof); purified magnesium aluminum silicates (commercially available as VEEGUM™ in various grades); purified sodium magnesium silicates (commercially available as LAPONITE™ in various grades); organically modified smectites including tetra alkyl and/or trialkyl ammonium smectites (organically modified montmorillonite clays) such as quaternium-18 bentonite, quaternium-18 hectorite, stearalkonium bentonite and stearalkonium hectorite; and mixtures thereof.

Montmorillonites represent clay minerals, which belong to the dioctahedral smectites, and are materials which swell in water but do not become plastic. The layer packets in the S-layer structure of the montmorillonites can swell as the result of reversible incorporation of water (in a 2-7 fold amount) and other substances, such as, for examples, alcohols, glycols, pyridine, α-picoline, ammonium compounds, hydroxyaluminosilicate ions, etc.

Since montmorillonite has a large capacity for ion exchange, aluminum can be replaced by Mg, Fe(II), Fe(III), Zn, Pb, Cr, Cu and others. The resulting negative Charge of the octahedral layers is balanced by cations, in particular Na⁺ (sodium montmorillonite) and Ca²⁺ (calcium montmorillonite) in interlayer positions.

The organophilization of montmorillonite or bentonites (exchange of the interlayer cations for quaternary alkylammonium ions) produces products (bentones), which are also useful herein.

The balance of the inorganic agent may be selected individually or as mixtures from the following: silicas, silicates, colloidal silicas, silicate pigments in which the free —OH (hydroxyl) groups on the surface of the particles have been (completely or partially) organically modified, chalk, talc, kaolin, Fullers earth, sodium polyacrylate, chemically modified magnesium aluminum silicate, hydrated aluminum silicate, zinc oxide, titanium oxide, and mixtures thereof.

Other Components

The formulations of the present invention may comprise a wide variety of additional components, as known in the art, including but not limited to anticaking agents, antimicrobial agents, astringents, opacifying agents, fragrances, pigments, preservatives, propellants, skin penetration enhancing agents, and waxes. See, for example, Harry's Cosmeticology, 7th Ed., Harry & Wilkinson (Hill Publishers, London 1982); Pharmaceutical Dosage Forms-Disperse Systems; Lieberman, Rieger & Banker, Vols. 1 (1988) & 2 (1989); Marcel Decker, Inc.; The Chemistry and Manufacture of Cosmetics, 2nd. Ed., deNavarre (Van Nostrand 1962-1965); and The Handbook of Cosmetic Science and Technology, 1st Ed. Knowlton & Pearce (Elsevier 1993).

Such additional components should be physically and chemically compatible with the vehicle and active components described herein, and not unduly impair stability, efficacy or other use benefits associated with the compositions of the present invention.

The compositions of the present invention are preferably formulated to have a pH of 10.5 or below, more preferably from about 3-8, and most preferably from about 5-8.

Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

In an exemplary, non-limiting general procedure, sunscreens, UV filters and other solids in the composition have to be dispersed in suitable solvents (in some instances, pre-dispersions of selected components, such as in a mineral oil or the like, are available to facilitate the procedure). Then, emollients, emulsifiers, lipophilic components (consistency factors) are combined and melted at an elevated temperature (e.g., from about 75° C. to about 95° C.). Stabilizers (thickeners) may then be dispersed separately under stirring in the water phase until homogeneous gel is formed and heated to 80° C. The two mixtures are combined progressively to form the emulsion, via mixing under intensive stirring until emulsion is formed. Gentle mixing continues while the emulsion is cooled. Sensitive components like the actives described herein (e.g., Cimicifuga extracts and ginsenosides), special additives, and preservatives should preferably be added after the mixture has been cooled (40-30° C.), in order to keep their properties intact.

Method for Regulating Skin Condition

The compositions of the present invention are useful for stimulating cellular growth, stimulating collagen production and/or stimulating fibronectin production in skin tissues, and/or promoting wound closure in skin tissues of a mammal, such as a human. Such increased cell growth and/or increased collagen or fibronectin production can help regulate or rejuvenate mammalian skin tissues. The compositions of the invention can be used for both prophylactic and therapeutic treatment of skin conditions. For example, compositions of the invention can be used for wound healing, thickening skin tissue (i.e., building the epidermis and/or dermis layers of the skin and where applicable the keratinous layers of the nail and hair shaft), preventing and/or retarding atrophy of mammalian skin, preventing and/or retarding the appearance of spider vessels and/or red blotchiness on mammalian skin, preventing and/or retarding the appearance of dark circles under the eye of a mammal, preventing and/or retarding sallow-colored mammalian skin, preventing and/or retarding sagging of mammalian skin, softening and/or smoothing lips, hair and nails of a mammal, preventing and/or relieving itch of mammalian skin, regulating skin texture (e.g. wrinkles and fine lines), and improving skin color (e.g. redness, freckles).

Treating skin tissues involves topically applying to the skin tissue a safe and effective amount of a composition of the present invention. The amount of the composition that is applied, the frequency of application and the period of use will vary widely depending e.g. upon the level of the ginseng extract, and/or Cimicifuga spp. Extract, or the optional genestein antioxidant, in a given composition and the level of regulation desired, for example, in light of the level of skin tissue damage present or expected to occur.

In some embodiments, the composition is chronically applied to the skin. The phrase “chronic topical application” as used herein refers to the continued topical application of the composition over an extended period during the subject's lifetime, for example, for a period of at least about one week, or for a period of at least about one month, or for at least about three months, or for at least about six months, or for at least about one year. While benefits are obtainable after various periods of use (e.g., two, five, ten or twenty days), chronic application can continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions that are typically applied per application are, in mg composition/cm² skin, from about 0.01 mg/cm² to about 10 mg/cm². A desirable and useful application amount is about 1 mg/cm² to about 2 mg/cm².

Regulating skin tissue condition can be practiced by applying a composition in the form of a skin lotion, cream, gel, foam, ointment, paste, emulsion, spray, conditioner, tonic, cosmetic, lipstick, foundation, nail polish, after-shave, or the like that is preferably intended to be left on the skin or other keratin structure for some esthetic, prophylactic, therapeutic or other benefit (i.e., a “leave-on” composition). After applying the composition to the skin, it can be left on the skin for a period of at least about 15 minutes, or at least about 30 minutes, or at least about 1 hour, or for at least several hours or, for example, at least about 12 hours.

Any part of the external portion of the face, hair, and/or nails can be treated, e.g., face, lips, gums, under-eye area, eyelids, scalp, neck, torso, arms, hands, legs, feet, fingernails, toenails, scalp hair, eyelashes, eyebrows, etc. The composition can be applied with the fingers or with an implement or device (e.g., pad, cotton ball, applicator pen, spray applicator, dental applicator and the like).

Another approach to ensure a continuous exposure of the skin to at least a minimum level of the beneficial compositions of the invention is to apply the composition in a patch, for example, to selected tissues such as the face. Such an approach is particularly useful for problem skin areas needing more intensive treatment (e.g., a wound, facial crows feet area, frown lines, under eye area, onto the gums and the like). The patch can be occlusive, semi-occlusive or non-occlusive and can be adhesive or non-adhesive. The composition can be contained within the patch or be applied to the skin prior to application of the patch. In a typical application the patch is preferably left on the skin for a period of at least about 5 minutes, or at least about 15 minutes, or at least about 30 minutes, or at least about 1 hour, or at night as a form of night therapy.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Preparation of Exemplary Anti-Aging Cream

A typical anti-aging cream of the present disclosure is formed by mixing and heating, as appropriate, the components recited in Table 1 (below), until the mixture is homogenous. The mixture may then be applied directly to the dermal surface of patients to reduce wrinkles, such as wrinkles on the face and hands.

TABLE I
Exemplary Formulation.

| | | ACTIVE OR |
|---|---|---|
| | AMOUNT | INACTIVE |
| INGREDIENT | (wt %) | COMPONENT |
| Trifolium pretense extract | 1.750 | Active |
| soy phytoestrogens¹ | 1.750 | Active |
| retinol palmitate | 0.250 | Active |
| Genestein | 0.100 | Active |
| Vitamin C palmitate² | 0.250 | Active |
| Vitamin B₅ | 1.000 | Active |
| Ginsenosides | 0.500 | Active |
| Arg-Gly-Asp tripeptide³ | 0.500 | Active |
| L-arginine | 1.500 | Active |
| L-glycine | 0.500 | Active |
| L-proline | 0.170 | Active |
| L-serine | 0.100 | Active |
| Cimicifuga racemosa extract | 0.200 | Active |
| Dexpanthenol | 1.00 | Active |
| D-panthenol | 1.00 | Active |
| Zinc gluconate | 0.100 | Active |
| Tocopherol acetate | 0.250 | Inactive |
| Heparin C30 | Trace | Inactive |
| Montmorillonite | 1.00 | Inactive |
| Glycerol | 2.00 | Inactive |
| almond oil | 1.50 | Inactive |
| Macadamia nut oil | 1.500 | Inactive |
| Moringa oleifera oil | 1.50 | Inactive |
| Hyaluronate sodium⁴ | 0.050 | Inactive |
| alpha-hydroxyl acid | Trace | Inactive |
| D.I. water | Remainder | Inactive |

¹glycine soja;
²L-ascorbic acid may be substituted;
³N-Acetyl D-glucosamine may be substituted;
⁴natural hyaluronal may be used equivalently.

Other and further embodiments utilizing one or more aspects of the inventions described above can be devised without departing from the spirit of Applicant's invention. For example, various specific extracts of the specific genus or species of plant, or chemical equivalents thereof, can be used in addition to, or in lieu of, the specific compounds listed herein. Further, the various methods and embodiments of the preparation and use of the compositions can be included in combination with each other to produce variations of the disclosed methods and embodiments. Discussion of Singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

What is claimed is:

1. A topical cosmetic, pharmaceutical, or dermatological composition comprising an effective quantity of L-arginine; at least one dermatologically acceptable compound comprising divalent zinc ions; and one or more extracts of a Panax, Cimicifuga, and/or a Trifolium species in a cosmetic vehicle.

2. The composition of claim 1, wherein the Panax species is selected from Panax Araliaceae, Panax bipinnatifidus, Panax ginseng, Panax japonicus, Panax quinquefolius, Panax trifolius, Panax vietnamensis, Panax wangianus, and Panax zingiberensis.

3. The composition of claim 1, wherein the Cimicifuga species is selected from C. racemosa, C. dahurica, C. foetida, and C. acerina.

4. The composition of claim 1, wherein the extract is from the root of the plant.

5. The composition of claim 1, further comprising at least one anti-oxidant and vitamin B₅ or a derivative thereof.

6. The composition of claim 1, further comprising an extract of an Astralagus species.

7. The composition of claim 1, further comprising stem cells of one or more plants.

8. The composition of claim 1, wherein the cosmetic vehicle in which the composition is present provides a gel, paste, cream, lotion, emulsion, or ointment comprising the composition.

9. A method for conditioning the skin of a mammal, the method comprising: applying topically to the skin a composition comprising an effective quantity of L-arginine; at least one dermatologically acceptable compound comprising divalent zinc ions; and one or more extracts of a Panax, Cimicifuga, and/or a Trifolium species in a cosmetic vehicle.

10. The method of claim 9, wherein the Panax species is selected from Panax Araliaceae, Panax bipinnatifidus, Panax ginseng, Panax japonicus, Panax quinquefolius, Panax trifolius, Panax vietnamensis, Panax wangianus, and Panax zingiberensis.

11. The method of claim 9, wherein the Cimicifuga species is selected from C. racemosa, C. dahurica, C. foetida, and C. acerina.

12. The method of claim 9, wherein the extract is from the root of the plant.

13. The method of claim 9, wherein the composition further comprises at least one anti-oxidant and vitamin B₅ or a derivative thereof.

14. The method of claim 9, wherein the composition further comprises an extract of an Astralagus species.

15. The method of claim 9, wherein the composition further comprises stem cells from one or more plants.

16. The method of claim 9, wherein the vehicle further comprises one or more additional ingredients selected from an emulsifier, a thickener, a skin emollient, and a inorganic particulate material.

17. The method of claim 9, wherein the cosmetic vehicle in which the composition is present provides a gel, paste, cream, lotion, emulsion, or ointment comprising the composition.

18. The method of claim 9, wherein the composition further comprises an epidermal skin cell activator, a collagen synthesis activator, a hyaluronic acid synthesis activator, a skin hydration activator, and a fibroblast migration activator, singly or in combination.
